(19) United States
(12) Patent Application Publication
(10) Pub. No.: US 2024/0151517 A1
(43) Pub. Date: May 9, 2024

(54) Shape Sensing of Multimode Optical Fibers

(71) Applicant: RAMOT AT TEL-AVIV UNIVERSITY LTD., Tel Aviv (IL)

(72) Inventors: Avishay Eyal, Givat Shmuel (IL); Alon Bahabad, Herzliya (IL); Daniel Marima, Tel Aviv (IL); Barak Hadad, Petah Tikva (IL); Nadav Magal, Tel Aviv (IL)

(21) Appl. No.: 18/548,939

(22) PCT Filed: May 15, 2022
(86) PCT No.: PCT/IB2022/054510

§ 371 Date: Sep. 5, 2023

Related U.S. Application Data
(60) Provisional application No. 63/192,121, filed on May 24, 2021.

(51) Int. Cl. G01B 11/24; A61B 34/20

(52) U.S. Cl. CPC G01B 11/2441; A61B 34/20; A61B 2034/2061; A61B 2090/3735

(57) ABSTRACT

A method for estimating a geometrical shape of an optical fiber (106) includes transmitting (102) into the optical fiber an input optical signal that excites in the optical fiber multiple propagation modes. An output optical signal, which is output from the optical fiber in response to the input optical signal, is recombined with a reference signal derived from the input optical signal, so as to produce a recombined optical signal. The recombined optical signal is detected (108), and the geometrical shape of the optical fiber (106) is estimated based on the detected recombined optical signal.

### CROSS-REFERENCE TO RELATED APPLICATIONS

This application claims the benefit of U.S. Provisional Patent Application 63/192,121, filed May 24, 2021, whose disclosure is incorporated herein by reference.

### FIELD OF THE INVENTION

The present invention relates generally to optical sensing, and particularly to methods and systems for acquisition and analysis of coherent optical signals from a multimode (MM) fiber.

### BACKGROUND OF THE INVENTION

A multimode (MM) optical fiber is an optical waveguide which supports multiple propagation modes which can be transmitted in the fiber with little loss. Each mode has its own propagation constant (wavenumber) and electromagnetic field distribution. The electromagnetic optical field distribution comprises information on the magnitude, phase and polarization distribution of the guided modes. When a beam of light is coupled into the fiber it may excite multiple modes in the fiber and they all will propagate in the fiber with, generally, different amplitudes and different propagation constants. The excitation amplitude of each mode depends on the overlap integral of the mode profile and the input beam. For ideal fiber which is straight and is not subject to external perturbations, the main effect which a mode experiences is phase accumulation. Still, there are coupling mechanisms between the various guided modes, e.g., due to Rayleigh scattering and residual birefringence, that cause amplitudes of modes to change as the modes propagate, but these effects are generally weak.

Despite their optical complexities, MM optical fibers are gaining traction in imaging and sensing. For example, Mingying Lan et al. report in a paper titled “Robust compressive multimode fiber imaging against bending with enhanced depth of field,” Optics Express, Vol. 27, No. 9, 29 April, 2019, 12957, how imaging through single multimode fiber prevails a counterpart using single mode fiber bundle on spatial resolution limit and minimum radius. Current multimode fiber imaging suffers from fussy calibration, which can be reduced by recent developed compressive sensing scheme. Experiments demonstrate improvement on depth of field by more than three orders of magnitude, together with robustness against macro fiber bending, which is vital to endoscopic applications.

Modeling of MM fibers has advanced to a point where an MM fiber can be modeled in a practical manner (e.g., in real time) despite the large number of guided modes supported by such fiber. For example, Shuhui Li et al. report in a paper titled “Compressively sampling the optical transmission matrix (TM) of a multimode fiber,” Light: Science & Applications (2021) 10:88, how optical measurement requirements can be relaxed using the framework of compressive sensing, in which the incorporation of prior information (e.g., an optical geometrical model) enables accurate estimation of the TM from fewer measurements than the dimensionality involved. This concept is demonstrated by reconstructing the full-size TM of a multimode fiber supporting 754 guided modes. The authors show that imaging is still possible using TMs reconstructed at very high compression ratios. Such approach offers a route towards the measurement of high dimensionality TMs, such as of MM fibers, either quickly or with access to limited numbers of measurements.

Deep learning (DL) methods were employed in the study of MM fibers. For example, Resisi et al. report in a paper titled “Image Transmission Through a Dynamically Perturbed Multimode Fiber by Deep Learning,” Laser and Photonics Reviews, Volume 15, Issue 10, October 2021, how when multimode optical fibers are perturbed, the data that is transmitted through them is scrambled. This presents a major difficulty for many possible applications, such as multimode fiber-based telecommunication and endoscopy. To overcome this challenge, a deep learning approach that generalizes over mechanical perturbations is presented. Using this approach, successful reconstruction of the input images from intensity-only measurements of speckle patterns at the output of a 1.5 m-long randomly perturbed multimode fiber is demonstrated. The model's success is explained by hidden correlations in the speckle of random fiber conformations.

### SUMMARY OF THE INVENTION

An embodiment of the present invention that is described hereinafter provides a method for estimating a geometrical shape of an optical fiber, the method including transmitting into the optical fiber an input optical signal that excites in the optical fiber multiple propagation modes. An output optical signal, which is output from the optical fiber in response to the input optical signal, is recombined with a reference signal derived from the input optical signal, so as to produce a recombined optical signal. The recombined optical signal is detected, and the geometrical shape of the optical fiber is estimated based on the detected recombined optical signal.

In some embodiments, the optical fiber does not contain any artificial structures for estimating the geometrical shape.

In some embodiments, detecting the recombined optical signal includes coherently detecting the recombined optical signal so as to produce multiple complex-valued images, the complex-valued images being indicative of respective field distributions over a cross-section of the optical fiber at respective positions along the optical fiber.

In an embodiment, estimating the geometrical shape includes deriving the geometrical shape for the respective positions from the complex-valued images.

In another embodiment, estimating the geometrical shape includes estimating a bending radius and a bending direction at a given position along the optical fiber, based on one or more complex-valued images obtained for respective positions in a vicinity of the given position.

In some embodiments, detecting the recombined optical signal includes registering a speckle pattern indicative of the geometrical shape of a section of the optical fiber, and estimating the geometrical shape includes deriving the geometrical shape of the section from the speckle pattern.

In some embodiments, transmitting the input optical signal includes transmitting an Optical Coherence Tomography (OCT) signal.

In an embodiment, estimating the geometrical shape includes applying a pre-trained neural network to the detected recombined optical signal.

In another embodiment, estimating the geometrical shape includes applying a transfer matrix (TM) process to the detected recombined optical signal.

In some embodiments, the optical fiber is part of a medical probe, and estimating the geometrical shape includes estimating a position of a distal end of the medical probe.

In some embodiments, the optical fiber is part of a building structure probe, and estimating the geometrical shape includes estimating structural changes over time.

In an embodiment, the optical fiber is used for transferring optical images independently of the input and output optical signals, and the method further includes compensating for distortion in transferal of the optical images based on the estimated geometrical shape.

There is additionally provided, in accordance with another embodiment of the present invention, an apparatus for estimating a geometrical shape of an optical fiber, the apparatus including an optical subsystem, and a processor. The optical subsystem is configured to transmit into the optical fiber an input optical signal that excites in the optical fiber multiple propagation modes, to recombine an output optical signal, which is output from the optical fiber in response to the input optical signal, with a reference signal derived from the input optical signal, so as to produce a recombined optical signal, and to detect the recombined optical signal. The processor is configured to estimate the geometrical shape of the optical fiber based on the detected recombined optical signal.

The present invention will be more fully understood from the following detailed description of the embodiments thereof, taken together with the drawings in which:

### BRIEF DESCRIPTION OF THE DRAWINGS

FIG. 1 is a block diagram that schematically illustrates a system for shape sensing of a multimode (MM) optical fiber, in accordance with an embodiment of the present invention; and

FIG. 2 is a flow chart that schematically describes a method of shape sensing of an MM optical fiber using the system of FIG. 1, in accordance with an embodiment of the present invention.

### DETAILED DESCRIPTION OF EMBODIMENTS

### Overview

Imaging using multimode (MM) fibers with core diameters ranging between several tens to several hundred microns, can be a preferred solution over a common solution of using a bundle made of numerous (e.g., tens of thousands) thin single-mode fibers. This may be particularly important for high-resolution medical image acquisition using endoscopes, in which, otherwise, a thick bundle of tens of thousands of fibers must be coupled, with high accuracy, to a pixelated camera, making the cost and dimensions of such an endoscope limiting factors, both commercially and clinically. Therefore, for example, imaging through an MM fiber can be important for the development of ultra-thin endoscopes.

However, when imaging through an MM fiber, the image transferred through such a fiber can be deciphered only if the fiber shape does not change (e.g., is kept straight), or if its shape is known, together with its transmission function for that shape. As the MM fiber of an endoscope is bound to move, fiber-shape sensing may well be required to enable optical imaging via such MM fibers. Shape sensing may also be very useful in cases where it is important to know the location of a specific part of the MM fiber in space, e.g., the end facet of an MM fiber, integrated with an endoscope, within the body of a patient. In another example, the fiber is part of a building structure probe, and estimating the geometrical shape of the fiber is used for estimating structural changes over time.

Obtaining sufficient image quality with MM fibers remains a challenge. A particularly important problem to overcome with MM fibers to maximize their potential as light guides is finding and compensating for changes in their shape (i.e., how they are bent and twisted along their length) by all-optical interrogation techniques.

One possible way for sensing the shape of an MM fiber is to inscribe Bragg gratings within the core or cladding of the fiber at various locations along the fiber and in its cross-section. When the fiber is bending to various directions the Bragg gratings are stressed, changing their periodicity and thus changing the spectral transmission and reflection of light going through them. The drawback of this method is the need for the fabrication of special fibers which makes them expensive.

Another possible solution for shape sensing is the use of light transmission through an MM fiber and inspecting the speckle image generated following transmission in the fiber. Machine learning (ML) techniques could be utilized to deduce the shape of the fiber by analyzing the speckle pattern. The performance of this sort of solution is very limited for very small modifications of the fiber shape. A few possible reasons account for such limited success. For example, it is not clear how well the information regarding the fiber shape is contained in the speckle pattern and whether there is a one-to-one mapping from a given shape to a respective speckle pattern.

Embodiments of the present invention that are described herein provide improved methods and systems for fiber shape sensing. The disclosed techniques are based on interrogation of a fiber using different spatial and/or polarization modes utilizing a broadband or narrowband light source. Different fiber shapes cause mode filtering, mode coupling, differential phase- and polarization-transformations due to effects induced by bending, geometric phase and birefringence effects, imprinting the fiber shape on the complex amplitudes, and polarization states of propagation modes. Analysis of these states can thus resolve the fiber shape.

The techniques described herein can be used with an MM fiber with no need for any additional optical components or structures within the MM fiber, such as Bragg gratings.

The methods and systems described herein rely on the following effects which occur when a fiber is bent at a given position:
Mode coupling: In the presence of mode coupling, light from a given mode is coupled to another mode. In this manner mode coupling redistributes the light energy between the various modes.
Loss to radiation modes (i.e., mode filtering): When the fiber is bent some light is coupled outside of the fiber, which contributes to loss. Generally, higher-order modes are more sensitive to loss induced by bending.
Changing wavenumbers of the modes at the location of the bending: As variations in wavenumbers are generally different for different modes, this leads to variations in the differential phase between the various modes.
Geometric phase can be added to modes when bending involves twisting out of plane (torsion).

The above mechanisms impart amplitude, phase- and polarization-transformations on different modes coupled to the fiber when it is twisted and turned. Further, these effects can be described with a linear relation between the fields at any two locations along the fiber. Thus, in principle, analysis of the linear transformation of the modes along the fiber can decipher the fiber's shape.

With an optical light source exciting the guided modes, it is possible, by using the disclosed technique, to characterize both the amplitude and phase of the optical field, at each location inside the MM fiber, with a typical spatial longitudinal resolution depending on the level of coherence, for example, on the order of a millimeter slice width AZ of the MM fiber. As an MM fiber for medical imaging experiences a bending radius of curvature on the order of a centimeter or more, shape sensing on a millimeter scale is sufficient.

To this end, with the collected optical data, an analytical approach known as a Transfer Matrix (TM) can retrieve the linear optical field \vec{E}(z) transformation along the MM fiber and learn its dependence on the local bending of the fiber. In this approach, complex-valued images (i.e., speckle images) are measured at predefined positions, z, along the MM fiber, for different spatial profiles and/or polarization states of the beam at the fiber input. The different beams correspond to excitation of different total optical fields \vec{E}(z) in the fiber. This is required in order to allow the calculation of a transfer matrix M(z), from which MM fiber geometry at a longitudinal location z (e.g., relative to a straight relaxed geometry) is analyzed.

In one embodiment, a wavelength swept source (i.e., tunable) narrowband laser source is used, and combining acquisitions made when the laser is being tuned over a wavelength range Δλ, corresponds to imaging a slice AZ of given width of, typically, a millimeter of the MM fiber. For example, if Δλ is sufficiently large (e.g., on the order of several nm about a center wavelength λ₀ of 1 μm), a complex-valued image of a slice of the MM fiber of the thickness ΔZ˜1 mm is obtained using an effective coherence length given by

$$l = \frac{{λ}_{0}^{2}}{Δλ} , and Δ z = l .$$

As further described below, with the analytic TM method many (e.g., tens of) measurements of the input and output of a fiber section (i.e., using the aforementioned data comprising complex-valued images acquired from locations along the MM fiber) are gathered to analyze and retrieve an optical transfer matrix of a fiber section depending on its local bending profile.

The above-described approach may use optical signals that are also used in Optical Coherence Tomography (OCT), albeit with an imaged depth orders of magnitude bigger than the typical depth in OCT. Accordingly, the spatial resolution is smaller. In addition, polarization measurements of different modes can be realized using polarizers with the system.

The coherent image (e.g., obtained with a frequency swept source laser) at any point along the fiber is a speckle pattern, which appears to be a random pattern of spots. As noted above, such an image is hard to interpret, since it is basically only one element of input information out of many required for a global fitting algorithm to the fiber shape over an entire length of the fiber. However, with a large enough number of modes interrogating the fiber, degeneracies in different fiber shape configurations on the speckle pattern can be removed to allow retrieving the shape of the fiber. ML techniques are used here to either find absolute fiber shapes, or the differential change between shapes, which can be used to monitor, in real time, the current shape of a fiber whose shape is manipulated over time.

In an alternative embodiment, a processor applies a deep learning (DL) algorithm to map collected optical intensity distribution to the fiber shape (i.e., speckle images), to learn the shape changes of the fiber in space. To this end, a processor uses a training set of acquired speckle images obtained under known conditions (e.g., known geometries of the MM fiber) to train a neural network (NN). Using the trained NN, the processor can infer newly acquired speckle images so as to describe an actual geometry of the MM fiber, including in real time.

For example, a narrowband laser source yields coherent speckle image with no localization (the coherent beam “samples” the effect of the entire length of the MM fiber). In this case, a direct approach for retrieving the local transformation of the field along the fiber is lost and a large set of speckle images is required to reflect multiple possible distortions of the MM fiber over its entire length. The bending and twisting of the fiber are imprinted on the speckle pattern of the field returning from the fiber. If enough modes are excited (with or without the excitation of different polarization states) possible degeneracies of speckle images with respect to fiber bending profiles are lifted and a ML approach can retrieve the fiber shape.

With a method for obtaining speckle images, an artificial neural network receives input pairs of images corresponding to the input and output of a given fiber section, and finds the bending radius and orientation at each longitudinal position. Both training and inference images are measured for many different spatial profiles and/or polarization states of the beam at the fiber input. The different beams correspond to excitation of different optical fields \vec{E}(z) in the fiber. This is required in order to allow the network to “find” M(z) (albeit not directly). Due to the local virtue of the optical measurement and deep learning implementation of a NN trained in a controlled experiment, it is expected that training the NN with bending at a given fiber location will readily generalize to all other fiber locations.

### System Description

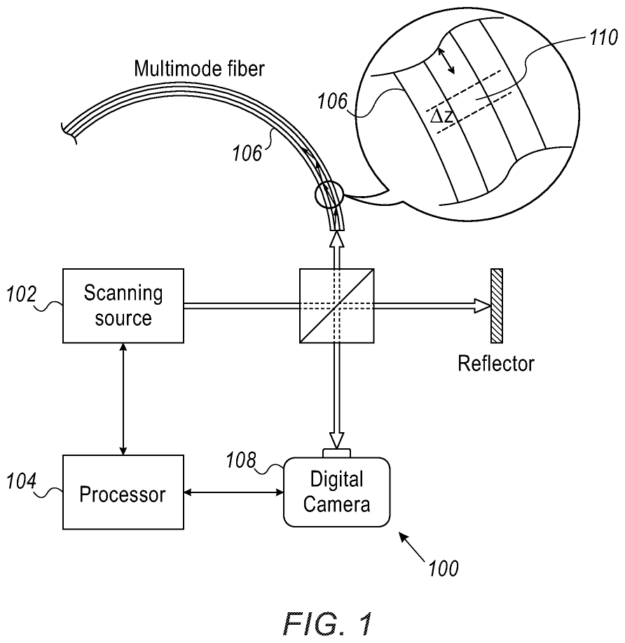

FIG. 1 is a block diagram that schematically illustrates a system 100 for shape sensing of a multimode (MM) optical fiber 106, in accordance with an embodiment of the present invention. System 100 has a layout of a Michaelson interferometer with a tunable laser source 102, its instantaneous frequency scanned in a manner similar to a swept source of an OCT system, such as a tunable laser at the 1.55 μm band with potentially tens of nanometers wavelength tuning range. In other embodiments, a broadband light source may be used. The source 102 may be modulated in beam profile (amplitude and phase) and polarization state, and is coupled into MM fiber 106 so that many modes of MM fiber 106 are excited. Either the optical field exiting the other side of the fiber, or the field propagating through the fiber and back, is mixed with a reference. In the shown example, a return light generated inside fiber 106 by Rayleigh scattering of the forward propagating beam, is combined by a beam-splitter with a reference beam obtained using a reflector.

The resulting combined beam is imaged by a digital camera 108. The measurements are frequency resolved (either by using a dispersion element in front of the camera or a frequency tunable laser source) which dictates a temporal resolution depending on the bandwidth of the source. This temporal resolution is directly translated to a spatial resolution ΔZ 110 along MM fiber 106, such as that which occurs in an OCT setup. Thus, a processor 104 controlling system 100 can retrieve images of the field (amplitude and/or phase and/or polarization) at specific points along the fiber, realizing a tomographic mapping (in a sense of imaging a slice of fiber 106 with an effective width of ΔZ 110) of the field in a multimode fiber.

If system 100 is applied in a global mode, after traversing the whole fiber (once, or twice following reflection from the distal end) the resulting image is a speckle pattern that characterizes the entire fiber length. As described above and below, such images can be used with deep learning techniques, as the information encoded in the speckle patterns is indirectly retrieved using statistical methods.

Different elements of system 100 may be implemented using suitable hardware, such as discrete components, one or more application-specific integrated circuits (ASICs) or field-programmable gate arrays (FPGAs), or in any other suitable way. In some embodiments, some elements of system 100 may be implemented using software, or using a combination of hardware and software elements. Elements of system 100 that are not relevant to understanding the disclosed techniques have been omitted from the figure for the sake of clarity.

As noted above, two interrogation approaches can be realized by system 100 of FIG. 1. In a local approach, each section of the fiber is interrogated locally. In a global approach the fiber shape as a whole is interrogated. The two approaches both use produce speckle images, albeit of different spatial resolution.

The acquisition regime is determined by an effective coherence length

$$∼ \frac{{λ}^{2}}{Δλ}$$

provided by source 102, that depends on the effective bandwidth Δλ in which the source is operated. For 1 nm sweeping the sampled fiber section length is about 1 mm, much shorter than typical fiber lengths on the order of 1 meter. This acquisition regime is a swept-source OCT like with slice resolution ΔZ of, for example, 1 mm, where the slice is acquired at a location z(t), according to the frequency of the interference combined from the reflections from the object (fiber) and reference.

Using a linewidth of the laser that is on an Angstrom scale, results in the coherence length at the IR regime of kilometers, much larger than typical fiber lengths, in endoscopic applications, on the order of 1 meter. As a result, the fiber geometry is interrogated as a whole, which requires to collect an ensemble of fiber geometries and identify the actual geometry using statistical or pattern recognition techniques such as of a trained NN. It should be therefore understood that multiple configurations of a fiber should be considered with each speckle pattern.

Using the analytical approach of a transfer matrix can retrieve from successive images, the linear field transformation along the fiber and learn its dependence on the local bending of the fiber, as described below.

The light at each position along the fiber can be described by using a vector \vec{E} whose elements are the complex amplitudes of all modes in the fiber which carry information regarding the power they carry and their phase.

As noted above, when a fiber is bent at a given position, several effects may occur, including mode coupling, loss to radiation modes, changes in mode wavenumbers, and addition of a geometric phase.

The effects induced by bending, and possibly twisting, modify an overall optical field \vec{E}. The modification of \vec{ } can be described using a z-dependent transmission matrix as:

\vec{E}(z+dz)=M(z)\vec{E}(z) (1.1)

The advantage of describing the bending induced variations using the matrix M(z) is due to it being a “local” entity. Namely, it describes the perturbed fiber properties at a given location, and it depends on the bending at this position alone. Accordingly, there is a one-to-one correspondence between M(z) and the bending radius and direction at each location. Hence, a measurement method which provides \vec{E}(z) at each position may be used to measure M(z) and, in turn, to yield the bending at points along the fiber.

### Measurement of \vec{E}(z)

In order to measure \vec{E}(z), a processor runs an algorithm employing the known method of Optical Coherence Tomography (OCT) which generates cross-sectional images of a transparent system with high resolution. For example, with swept-source OCT (SS-OCT) the multimode fiber is connected to the sensing arm of an optical Michelson interferometer, as described in FIG. 1. Light from a scanning-frequency laser, such as source 102, is split using a beam splitter between the sensing arm and a reference arm. Light which propagates in the sensing fiber experiences backscatter at each position along the fiber due to Rayleigh scattering. The backscattered light is recombined with a reference and the resulting image is detected via a digital camera. The laser's frequency is scanned according to fᵢₙₛₜ(t)=f₀+γt, f(t)=c/λ(t). The signal of each pixel in the captured image has a time dependence of:

V(x,y,t)˜∫R(x,y,τ)exp{j[2πγτt+ϕ(x,y,τ)]}dτ+{negative_frequency_terms} (1.2)

where τ describes the differential roundtrip time to a given position in the fiber and is directly related to z. A (time-domain) Fourier transform of V(x,y,t) yields R(x,y,τ)exp[jϕ(x,y,τ)] which can be viewed as a transformed version of \vec{E}(z). Namely, each position in the fiber (identified by its unique roundtrip delay τ) reveals a complex backscattered image. SS-OCT is but one possible option. Any other known variant of the method, such as spectrometer-based OCT (SB-OCT) in which a broadband light source is used and a diffraction grating disperse the light returning from the fiber into a detector array, or a time-domain OCT (TD-OCT) employing a suitable light source, can be used. Yet another option is polarization-sensitive OCT (PS-OCT) which may prove useful, since the bending of the fiber changes the polarization state of light propagating in the fiber.

### ML Methods in Shape Sensing Using a Global Approach

An alternative approach is transmitting, into the fiber, a set of modes characterized by different spatial profiles and/or different polarizations, and then registering the speckle pattern gathered for light output from the fiber. The effects caused by the overall bending profile of the fiber is imprinted on this speckle pattern. As noted above, with a large enough number of modes interrogating the fiber, degeneracies in different fiber-shape configurations on the speckle pattern can be lifted to allow retrieving the shape of the fiber. Deep learning is here used to either find absolute fiber shapes or the differential change between shapes which can be used, in real time, to monitor the current shape of a fiber whose shape is manipulated over time.

Assuming that a large enough cohort of speckle images is obtained with a sufficiently large set of relevant fiber deformations, trained DL techniques can be used to either find absolute fiber 106 shapes, or the differential change between shapes, which could be used, in real time, to monitor the current shape of MM fiber 106 whose shape is manipulated over time (e.g., while in use in an endoscope).

### Methods of Shape Sensing of Multimode Optical Fibers

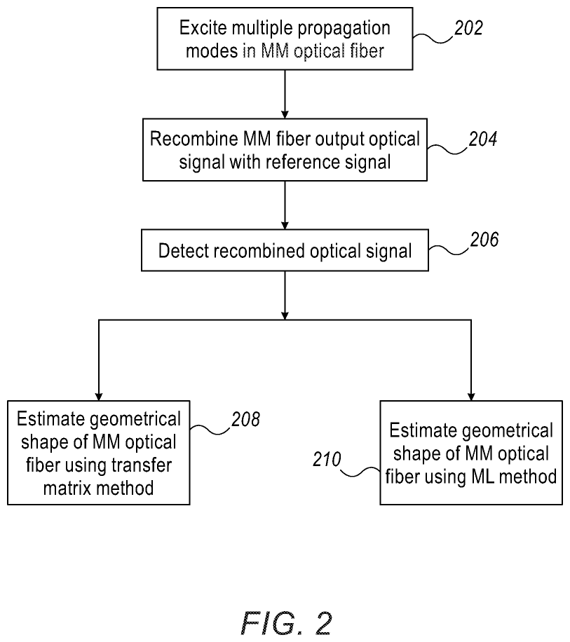

FIG. 2 is a flow chart that schematically describes a method of shape sensing of an MM optical fiber using system 100 of FIG. 1, in accordance with an embodiment of the present invention. The algorithm, according to the presented embodiment, carries out a process that begins with processor 104 using source 102 to excite multiple (e.g., thousands) propagation modes in MM fiber 106, at modes excitation step 202.

The interferometric system recombines the backscatter optical output of MM fiber 106 with a reference signal, at signal recombining step 204.

Digital camera 108 detects (e.g., images) the recombined signal, at recombined signal detection step 206. The image captured by camera 108 as a function of time can be Fourier transformed (in the temporal variable) to yield a series of images, each related to a specific depth.

Integrating over dummy variable τ (where τ is determined by the length of the fiber), as modeled in eq. 1.2, is a property of the acquisition, and thus takes place automatically, so to speak.

Next, processor 102 can apply an analytic TM method to analyze the shape of MM fiber 106 from the complex-value images, at an analytical shape sensing step 208. Alternatively, processor 102 can apply an ML method to analyze the shape of MM fiber 106 from the speckle images, at an ML based shape sensing step 210. Moreover, processor 102 can even apply a combination of both steps 208 and 210 to analyze the optical data.

The flow chart of FIG. 2 is brought by way of example and is simplified to maintain clarity of presentation. Other steps may be included in the process, such as optical and/or electronic filtration.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A method for estimating a geometrical shape of an optical fiber, the method comprising:
transmitting into the optical fiber an input optical signal that excites in the optical fiber multiple propagation modes;
recombining an output optical signal, which is output from the optical fiber in response to the input optical signal, with a reference signal derived from the input optical signal, so as to produce a recombined optical signal; and
detecting the recombined optical signal, and estimating the geometrical shape of the optical fiber based on the detected recombined optical signal.

2. The method according to claim 1, wherein the optical fiber does not contain any artificial structures for estimating the geometrical shape.

3. The method according to claim 1, wherein detecting the recombined optical signal comprises coherently detecting the recombined optical signal so as to produce multiple complex-valued images, the complex-valued images being indicative of respective field distributions over a cross-section of the optical fiber at respective positions along the optical fiber.

4. The method according to claim 3, wherein estimating the geometrical shape comprises deriving the geometrical shape for the respective positions from the complex-valued images.

5. The method according to claim 3, wherein estimating the geometrical shape comprises estimating a bending radius and a bending direction at a given position along the optical fiber, based on one or more complex-valued images obtained for respective positions in a vicinity of the given position.

6. The method according to claim 1, wherein detecting the recombined optical signal comprises registering a speckle pattern indicative of the geometrical shape of a section of the optical fiber, and wherein estimating the geometrical shape comprises deriving the geometrical shape of the section from the speckle pattern.

7. The method according to claim 1, wherein transmitting the input optical signal comprises transmitting an Optical Coherence Tomography (OCT) signal.

8. The method according to claim 1, wherein estimating the geometrical shape comprises applying a pre-trained neural network to the detected recombined optical signal.

9. The method according to claim 1, wherein estimating the geometrical shape comprises applying a transfer matrix (TM) process to the detected recombined optical signal.

10. The method according to claim 1, wherein the optical fiber is part of a medical probe, and wherein estimating the geometrical shape comprises estimating a position of a distal end of the medical probe.

11. The method according to claim 1, wherein the optical fiber is part of a building structure probe, and wherein estimating the geometrical shape comprises estimating structural changes over time.

12. The method according to claim 1, wherein the optical fiber is used for transferring optical images independently of the input and output optical signals, and wherein the method further comprises compensating for distortion in transferal of the optical images based on the estimated geometrical shape.

13. An apparatus for estimating a geometrical shape of an optical fiber, the apparatus comprising:
an optical subsystem, configured to transmit into the optical fiber an input optical signal that excites in the optical fiber multiple propagation modes, to recombine an output optical signal, which is output from the optical fiber in response to the input optical signal, with a reference signal derived from the input optical signal, so as to produce a recombined optical signal, and to detect the recombined optical signal; and
a processor, configured to estimate the geometrical shape of the optical fiber based on the detected recombined optical signal.

14. The apparatus according to claim 13, wherein the optical fiber does not contain any artificial structures for estimating the geometrical shape.

15. The apparatus according to claim 13, wherein the optical subsystem is configured to coherently detect the recombined optical signal so as to produce multiple complex-valued images, the complex-valued images being indicative of respective field distributions over a cross-section of the optical fiber at respective positions along the optical fiber.

16-17. (canceled)

18. The apparatus according to claim 13, wherein, in detecting the recombined optical signal, the optical subsystem is configured to register a speckle pattern indicative of the geometrical shape of a section of the optical fiber, and wherein the processor is configured to derive the geometrical shape of the section from the speckle pattern.

19. (canceled)

20. The apparatus according to claim 13, wherein the processor is configured to estimate the geometrical shape by applying a pre-trained neural network or a transfer matrix (TM) process to the detected recombined optical signal.

21. (canceled)

22. The apparatus according to claim 13, wherein the optical fiber is part of a medical probe, and wherein the processor is configured to estimate a position of a distal end of the medical probe.

23. The apparatus according to claim 13, wherein the optical fiber is part of a building structure probe, and wherein the processor is configured to estimate structural changes over time.

24. The apparatus according to claim 13, wherein the optical fiber is used for transferring optical images independently of the input and output optical signals, and wherein the processor is further configured to compensate for distortion in transferal of the optical images based on the estimated geometrical shape.
